(19) United States
(12) Patent Application Publication
(10) Pub. No.: US 2023/0064561 A1
(43) Pub. Date: Mar. 2, 2023

(54) SYSTEMS AND METHODS OF DETECTING HORMONES OR ANALYTES FOR FERTILITY AND PREGNANCY MONITORING

(71) Applicant: MFB Fertility, Inc., Boulder, CO (US)

(72) Inventor: Amy BECKLEY, Erie, CO (US)

(73) Assignee: MFB Fertility, Inc., Boulder, CO (US)

(21) Appl. No.: 17/897,132

(22) Filed: Aug. 27, 2022

Related U.S. Application Data
(60) Provisional application No. 63/238,130, filed on Aug. 28, 2021.

(51) Int. Cl. A61B 10/00; A61B 5/145; A61B 5/1473; A61B 5/00

(52) U.S. Cl. CPC A61B 10/0012; A61B 5/14514; A61B 5/14735; A61B 5/685; A61B 5/14546; A61B 2010/0016

(57) ABSTRACT

Provided herein are systems and methods that use an electrochemical aptamer-based (EAB) biosensor to detect a plurality of hormone and/or analyte that can be used for fertility and pregnancy monitoring.

### CROSS-REFERENCE TO RELATED APPLICATION

This application claims the priority to and benefit of U.S. Provisional Application 63/238,130 filed on Aug. 28, 2021, the entire content of which is incorporated herein by reference for all purposes.

### FIELD

The present disclosure relates generally to systems and methods for fertility and pregnancy monitoring, and more specifically to systems and methods that use microneedle arrays for biosensing hormones and/or analytes for fertility and pregnancy monitoring.

### BACKGROUND

Fertility is largely governed by the levels of certain hormones associated with the menstrual cycle. For example, a rise in luteinizing hormone (LH) will stimulate release of the oocyte, or egg, and is typically indicative of the ovulation window. Similarly, an increase in human chorionic gonadotropin (HCG) hormone can signal that pregnancy has occurred. For this reason, evaluating hormonal changes can serve as a way for assessing and predicting fertility. However, a major hurdle to this method of fertility tracking is the need to monitor levels of multiple hormones over an extended period time. While urine tests that measure the levels of hormones or their metabolites do exist, such tests do not provide continuous monitoring of hormone levels. Furthermore, hormones in the bloodstream may not materialize instantly in the urine. Current methods of blood serum testing are also inefficient in that they require laboratory analysis, which is costly, takes a longer time to complete and, again only provides a read out of hormone levels at a given point in time which has passed.

Thus, what is desirable in the art is to provide systems for and methods that can continually track hormones and/or analytes for fertility and pregnancy monitoring.

### BRIEF SUMMARY

In some aspects, provided herein are systems and methods that can continuously monitoring levels of multiple hormones and/or analytes at the same time.

In certain aspects, provided is a system for detecting hormones and/or analytes in interstitial fluid in a subject. In some embodiments, the system includes: an electrochemical aptamer-based (EAB) biosensor; a means for providing a potential to facilitate an electrochemical reaction associated with at least one of the aptamers; and a means to detect and communicate a result. In some variations, the EAB biosensor includes at least one hormone and/or analyte sensing microneedle array configured to access interstitial fluid in the subject. The EAB sensor is configured for in vivo detection of a plurality of hormones and/or analytes in the interstitial fluid, and to generate one or more signals associated with the detection of at least one of the plurality of hormones and/or analytes. In some variations, the microneedle array comprises a plurality of microneedles, in which one or more of the microneedles have a coating to form an interrogating electrode that is configured to insert into intradermal space of the subject; an aptamer is immobilized on the surface of the interrogating electrode; and a redox reporter is linked to the aptamer. In certain variations of the foregoing, at least one of the microneedles in the microneedle array comprises an aptamer that selectively binds to luteinizing hormone (LH). In other variations of the foregoing, at least one of the microneedles in the microneedle array comprises an aptamer that selectively binds to luteinizing hormone (LH), and at least one of the microneedles in the microneedle array comprises an aptamer that selectively binds to progesterone, or a metabolite thereof. In certain embodiments, the EAB sensor further includes a reference electrode having a site of known redox potential; and a counter electrode configured to act as a cathode or anode to the interrogating electrode.

In certain aspects, provided are also methods for using the systems described herein for fertility and pregnancy monitoring, including, for example, for predicting and/or confirming ovulation of a subject. In some embodiments, the method includes: measuring at least one hormone and/or analyte from interstitial fluid of the subject; and measuring a concentration change of luteinizing hormone (LH), or progesterone or a metabolite thereof, or any combination thereof in the subject. In some variations, at least one hormone and/or analyte is LH, progesterone, estrogen, or follicle stimulating hormone (FSH), or any metabolite thereof, or any combination of the foregoing.

In other embodiments, the method includes: determining a baseline luteinizing hormone level in the subject prior to ovulation by continuous monitoring average luteinizing hormone levels over an initial time period of at least 10 minutes, wherein the initial time period is cycle start date until 18 days before the next expected cycle start date; and predicting ovulation when the luteinizing hormone levels: a) increase at least 2-fold over the baseline luteinizing hormone level; and/or b) reach an absolute luteinizing hormone level of greater than 20 mUI/ml.

In yet other embodiments, the method includes: determining a baseline of progesterone level, or a level of metabolites thereof, in the subject prior to ovulation by continuous monitoring average progesterone levels, or average levels of metabolites thereof, over an initial time period of at least 10 minutes, wherein the initial time period is cycle start date until 18 days before the next expected cycle start date; and confirming ovulation when progesterone levels, or levels of metabolites thereof: a) increase at least 2-fold over the baseline of progesterone level, or a level of metabolites thereof, and/or b) reach an absolute progesterone level, or level of metabolites thereof, of greater than 5 ng/ml progesterone or a metabolite thereof.

In one aspect, provided is a method of confirming pregnancy and monitoring the health of a pregnancy in a subject using any of the systems described herein. In some embodiments, the method includes: measuring HCG, progesterone or a metabolite thereof, and/or luteinizing hormone (LH); and a) calculating a conception date based on 1) the date in which LH was above 20 mUI/ml, 2) LH levels reach 2- or 3-fold above baseline values, 3) the date progesterone or a metabolite thereof levels reached 2-fold over baseline values, and/or 4) the range of dates between LH rise and rise in progesterone or a metabolite thereof as a possible conception window; and/or b) assessing health of pregnancy by calculating 1) the rate at which HCG increases, 2) measuring progesterone or a metabolite thereof levels continuously to determine the rate at which progesterone or a metabolite thereof levels increase, to alert the subject when levels of progesterone or a metabolite thereof fall below 5 ng/ml, 10 ng/ml, or 15 ng/ml, and/or 3) drop in progesterone signals need to support pregnancy with additional progesterone supplementation.

### DETAILED DESCRIPTION

The following description sets forth exemplary systems, methods, parameters and the like. It should be recognized, however, that such description is not intended as a limitation on the scope of the present disclosure but is instead provided as a description of exemplary embodiments.

In some aspects, provided is a system for detecting hormones and/or analytes in interstitial fluid in a subject. In some variations, the subject is a human and female. In certain variations, the subject is an adult. The systems provided herein use an electrochemical aptamer-based (EAB) biosensor to detect a plurality of hormone and/or analyte that can be used for fertility and pregnancy monitoring.

In certain aspects, provided are also methods using any of the systems described herein for predicting and/or confirming ovulation, as well as confirming pregnancy and monitoring the health of a pregnancy. For example, the systems can be used to measure luteinizing hormone (LH), progesterone, or a metabolite thereof (such as pregnanediol), and human chorionic gonadotropin (HCG) to predict ovulation or optimal conception; to determine the moment of conception by monitoring HCG; and to track the health of the pregnancy by monitoring levels of progesterone, or a metabolite thereof and the doubling rate of HCG. In the case of early pregnancy, HCG should double every 48 hours.

### EAB Biosensors

The EAB biosensor is typically a three-electrode cell, which includes an interrogating electrode, a reference electrode, and a counter electrode.

Interrogating Electrode

In some embodiments, the EAB biosensor includes at least one hormone and/or analyte sensing microneedle array configured to access interstitial fluid in the subject. In some variations, the EAB sensor is configured for in vivo detection of a plurality of hormones and/or analytes in the interstitial fluid, and to generate one or more signals associated with the detection of at least one of the plurality of hormones and/or analytes. The microneedle array comprises a plurality of microneedles. One or more of the microneedles have a coating to form the interrogating electrode that is configured to insert into intradermal space of the subject. Suitable coatings may include, for example, gold, silver and/or carbon.

In some variations, the microneedle has a length between 0.05 mm to 4.0 mm, or between 0.5 mm and 1 mm. In some embodiments the microneedle is made of silicon crystal. In certain embodiments, the silicon crystal is pure. In some variations, the microneedle incorporates microelectromechanical or micro-electro-mechanical systems (MEMS). In some embodiments, the microneedle is FDA certified, CE certified and/or ISO 13485 certified.

Each microneedle has an inner wall and an outer wall. The microneedle(s) may have a hollow interior or may be solid.

In some variations, at least a portion of the microneedles has a hollow interior. In such variation, the microneedle also has an opening at the terminal end that exposes the hollow interior. In one variation, the inner wall of the hollow microneedle is coated. In another variation, the outer wall of the hollow microneedle is coated. When the inner wall of the microneedle is coated, the interrogating electrode is positioned inside the microneedle.

In other variations, at least a portion of the microneedles has a solid interior, and the outer wall of the microneedle is coated. When the outer wall of the microneedle is coated, the interrogating electrode is positioned outside the microneedle. In both instances, the portion of the microneedle that has the interrogating electrode inserts into intradermal space of the subject, thereby providing in vivo detection.

In some embodiments, an aptamer is immobilized on the surface of the interrogating electrode. For example, in one variation, the aptamer is immobilized on the surface of the interrogating electrode via a thiol group. The thiol group may be covalently bonded to the interrogating electrode.

In some embodiments, a redox reporter is linked to the aptamer. Suitable examples of the redox reporting may include, for example, methylene blue, ferrocene, or silver nanoparticles, or any combination thereof.

In certain embodiments, at least one of the microneedles in the microneedle array comprises an aptamer that selectively binds to luteinizing hormone (LH). In certain embodiments, (i) at least one of the microneedles in the microneedle array comprises an aptamer that selectively binds to luteinizing hormone (LH), and (ii) at least one of the microneedles in the microneedle array comprises an aptamer that selectively binds to progesterone, or a metabolite thereof. In some variations of the foregoing, at least one of the microneedles that comprises an aptamer that selectively binds to HCG, one or more estrogen metabolites or estrones, vitamin D, testosterone, or cortisol. For example, in one variation, (i) at least one of the microneedles in the microneedle array comprises an aptamer that selectively binds to luteinizing hormone (LH), (ii) at least one of the microneedles in the microneedle array comprises an aptamer that selectively binds to progesterone, or a metabolite thereof, and (iii) at least one of the microneedles that comprises an aptamer that selectively binds to HCG, one or more estrogen metabolites or estrones, vitamin D, testosterone, or cortisol.

In some variations, the aptamer is a nucleic acid. In certain variations, the aptamer is a DNA or RNA molecule. In some variations, the aptamer is of a full DNA or RNA sequence. In other variations, the aptamer is of a truncated DNA or RNA sequence. In certain variations, the aptamer is a 10-50 base pair or 20-30 base pair DNA or RNA molecule. In one variation, the aptamer is biotinylated.

In some embodiments, the EAB sensor may be configured to monitor up to nine hormones and/or analytes at once. Such hormones and/or analytes may include, for example, luteinizing hormone (LH), progesterone, human chorionic gonadotropin (HCG), cortisol, testosterone, anti-mullerian hormone (AMH), prolactin, dehydroepiandrosterone (DHEA, also known as androstenolone), estrogen, Estrone-3-Glucuronide (E1G) or other estrogen metabolite, pregnanediol-3-glucuronide (PDG, progesterone metabolite), and thyroid-stimulating hormone (TSH).

In some variations, suitable aptamers that selectively bind to LH include, for example, a B23 aptamer. In some variations, suitable aptamers that selectively bind to progesterone, or a metabolite thereof, include, for example, P4G13 aptamer. In some variations, suitable aptamers that selectively bind to HCG may include, for example, a 19-C09 aptamer.

In some embodiments, a stabilizer may be used to stabilize the aptamers. For example, the stabilizer may include a buffer. In one variation, the stabilizer may include sugars.

Reference Electrode and Counter Electrode

The reference electrode includes a site of a known chemical reaction that has a known redox potential, which is used as the measuring point for the redox potential of the interrogating electrode. The counter electrode acts as a cathode or anode to the interrogating electrode.

Current is measured as potential of the interrogating electrode as compared the fixed potential of the reference electrode. The difference in potential is what generates a signal.

Other System Components

In some embodiments, the system further includes a means for providing a potential to facilitate an electrochemical reaction associated with at least one of the aptamers, and a means to detect and communicate a result. In some variations, the potential applied to the system is generated by a circuit board and battery communicatively connected to the interrogating electrode.

In some embodiments, the array of microneedles further comprises a means for near-field communications energy harvesting for providing the potential.

In some embodiments, the system further includes a means for communicating a detected quantitative hormone value result and displaying the result upon a user interface. In some variations, the means for providing a potential to facilitate an electrochemical reaction associated with at least one of the aptamers, the means to detect and communicate a result, and the means to display the result are part of an external unit.

In some embodiments, the system further includes a means to control a current to pass no more often than every ten minutes.

In some embodiments, the system further includes a means to store the information/results associated with the applied potential and correlate to the time that the scan was taken.

In some embodiments, the system is configured to discern channels and associate each channel with a result for LH, progesterone or a metabolite thereof, HCG, one or more estrogen metabolites or estrones, vitamin D, testosterone, or cortisol.

In some embodiments, the system further includes a housing. Any suitable materials may be used for the housing. For example, in some variations, the housing comprises a plastic material or an adhesive material. Any suitable combination of materials may also be used for the housing.

In some embodiments, the system is a disposable wearable device. The disposal wearable device may be placed on any suitable body part of the subject, for example, on the abdomen of the subject.

### Methods of Use

In some aspects, provided is method of predicting and/or confirming ovulation of a subject using any of the systems described herein. In some embodiments, the method includes: measuring at least one hormone and/or analyte from interstitial fluid of the subject; and measuring a concentration change of luteinizing hormone (LH), or progesterone or a metabolite thereof, or any combination thereof in the subject. In some variations, the hormones and/or analytes may include, for example, LH, progesterone, estrogen, or follicle stimulating hormone (FSH), or any metabolite thereof, or any combination of the foregoing.

In some variations, the method further includes using at least two urine-based LH test strips to indicate a time for use of the system, and wherein the system continuously measures at least LH for at least 6 hours.

In certain variations, the method includes continuously measuring at least LH for at least 24 hours. In other embodiments, the method further includes taking at least one measurement of progesterone, or a metabolite thereof (such as pregnanediol). In certain variations, the method includes measuring both LH and progesterone for at least 6 hours.

In some variations, the method further includes providing continuous concentration data of at least one hormone and/or analyte measured, a predicted time of ovulation, a predicted time of peak fertility, or a confirmation that ovulation has occurred, or any combination thereof. In other variations, the method further includes providing continuous concentration data of at least one hormone and/or analyte measured, a predicted time of ovulation, a predicted time of peak fertility, and a confirmation that ovulation has occurred, or any combination thereof.

In one aspect, provided is a method for predicting ovulation in a subject using any of the systems described herein. In some embodiments, the method includes: determining a baseline luteinizing hormone level in the subject prior to ovulation by continuous monitoring average luteinizing hormone levels over an initial time period of at least 10 minutes, wherein the initial time period is cycle start date until 18 days before the next expected cycle start date; and predicting ovulation when the luteinizing hormone levels: a) increase at least 2-fold over the baseline luteinizing hormone level; and/or b) reach an absolute luteinizing hormone level of greater than 20 mUI/ml. In some variations, ovulation is predicted when the luteinizing hormone levels increase 2-fold or 3-fold over the baseline luteinizing hormone level.

In another aspect, provided is a method of confirming ovulation in a subject using any of the systems described herein. In some embodiments, the method includes: determining a baseline of progesterone level, or a level of metabolites thereof, in the subject prior to ovulation by continuous monitoring average progesterone levels, or average levels of metabolites thereof, over an initial time period of at least 10 minutes, wherein the initial time period is cycle start date until 18 days before the next expected cycle start date; and confirming ovulation when progesterone levels, or levels of metabolites thereof: a) increase at least 2-fold over the baseline of progesterone level, or a level of metabolites thereof, and/or b) reach an absolute progesterone level, or level of metabolites thereof, of greater than 5 ng/ml progesterone or a metabolite thereof. In some variations, ovulation is predicted when the progesterone levels, or levels of metabolites thereof, increase 2-fold or 3-fold over the baseline progesterone level, or level of metabolites thereof. In one variation, the metabolite of progesterone is pregnanediol. In certain embodiments, the initial time period is at least 2 hours, or about 2 hours.

In yet another aspect, provided is a method of confirming pregnancy and monitoring the health of a pregnancy in a subject using any of the systems described herein. In some embodiments, the method includes: measuring HCG, progesterone or a metabolite thereof, and/or luteinizing hormone (LH); and a) calculating a conception date based on 1) the date in which LH was above 20 mUI/ml, 2) LH levels reach 2- or 3-fold above baseline values, 3) the date progesterone or a metabolite thereof levels reached 2-fold over baseline values, and/or 4) the range of dates between LH rise and rise in progesterone or a metabolite thereof as a possible conception window; and/or b) assessing health of pregnancy by calculating 1) the rate at which HCG increases (e.g., ideally HCG doubles in 24-72 hours the first 6 weeks of pregnancy), 2) measuring progesterone or a metabolite thereof levels continuously to determine the rate at which progesterone or a metabolite thereof levels increase, to alert the subject when levels of progesterone or a metabolite thereof fall below thresholds, such as 5 ng/ml, 10 ng/ml, or 15 ng/ml, and/or 3) drop in progesterone signals need to support pregnancy with additional progesterone supplementation.

What is claimed is:

1. A system for detecting hormones and/or analytes in interstitial fluid in a subject, comprising:
an electrochemical aptamer-based (EAB) biosensor, comprising at least one hormone and/or analyte sensing microneedle array configured to access interstitial fluid in the subject, wherein the EAB sensor is configured for in vivo detection of a plurality of hormones and/or analytes in the interstitial fluid, and to generate one or more signals associated with the detection of at least one of the plurality of hormones and/or analytes,
wherein the microneedle array comprises a plurality of microneedles,
wherein one or more of the microneedles have a coating to form an interrogating electrode that is configured to insert into intradermal space of the subject,
wherein an aptamer is immobilized on the surface of the interrogating electrode, and
wherein a redox reporter is linked to the aptamer, and
wherein:
(i) at least one of the microneedles in the microneedle array comprises an aptamer that selectively binds to luteinizing hormone (LH), or
(ii) at least one of the microneedles in the microneedle array comprises an aptamer that selectively binds to luteinizing hormone (LH), and at least one of the microneedles in the microneedle array comprises an aptamer that selectively binds to progesterone, or a metabolite thereof;
a means for providing a potential to facilitate an electrochemical reaction associated with at least one of the aptamers; and
a means to detect and communicate a result.

2. The system of claim 1, wherein the EAB sensor further comprises:
a reference electrode having a site of known redox potential; and
a counter electrode configured to act as a cathode or anode to the interrogating electrode.

3. The system of claim 1, wherein the microneedles comprise pure silicon crystal.

4. The system of claim 1, wherein at least a portion of the microneedles has a hollow interior.

5. The system of claim 4, wherein the microneedle has an inner wall and an outer wall, and wherein the inner wall of the microneedle is coated.

6. The system of claim 4, wherein the microneedle has an inner wall and an outer wall, and wherein the outer wall of the microneedle is coated.

7. The system of claim 1, wherein at least a portion of the microneedles has a solid interior.

8. The system of claim 7, wherein the microneedle has an inner wall and an outer wall, and wherein the outer wall of the microneedle is coated.

9. The system of claim 1, wherein the electrode comprises gold, silver, or carbon, or any combination thereof.

10. The system of claim 1, wherein the aptamer is immobilized on the surface of the electrode via a thiol group.

11. The system of claim 10, wherein the aptamer is covalently bonded to the electrode via the thiol group.

12. The system of claim 1, wherein at least one of the microneedles in the microneedle array comprises an aptamer that selectively binds to HCG, one or more estrogen metabolites or estrones, vitamin D, testosterone, or cortisol.

13. The system of claim 1, wherein the aptamer that selectively binds to LH comprises a B23 aptamer.

14. The system of claim 1, wherein the aptamer that selectively binds to progesterone comprises a P4G13 aptamer.

15. The system of claim 12, wherein the aptamer that selectively binds to HCG comprises a 19-C09 aptamer.

16. The system of claim 1, wherein the redox reporter comprises methylene blue, ferrocene, or silver nanoparticles, or any combination thereof.

17. The system of claim 1, wherein the potential applied to the system is generated by a circuit board and battery communicatively connected to the interrogating electrode.

18. The system of claim 1, wherein the array of microneedles further comprises a means for near-field communications energy harvesting for providing the potential.

19. The system of claim 18, further comprising: a means for communicating a detected quantitative hormone value result and displaying the result upon a user interface.

20. The system of claim 19, wherein the means for providing a potential to facilitate an electrochemical reaction associated with at least one of the aptamers, the means to detect and communicate a result, and the means to display the result are part of an external unit.

21. The system of claim 1, further comprising: a means to control a current to pass no more often than every ten minutes.

22. The system of claim 1, further comprising: a means to store the information/results associated with the applied potential and correlate to the time that the scan was taken.

23. The system of claim 1, wherein the system is configured to discern channels and associate each channel with a result for LH, progesterone or a metabolite thereof, HCG, one or more estrogen metabolites or estrones, vitamin D, testosterone, or cortisol.

24. The system of claim 1, further comprising: a housing.

25. The system of claim 24, wherein the housing comprises a plastic material or an adhesive material.

26. The system of claim 1, wherein the system is a disposable wearable device.

27. The system of claim 26, wherein the disposable wearable device is configured for placement on the abdomen of the subject.

28. A method of predicting and/or confirming ovulation of a subject using a system of claim 1, the method comprising;
measuring at least one hormone and/or analyte from interstitial fluid of the subject; and
measuring a concentration change of luteinizing hormone (LH), or progesterone or a metabolite thereof, or any combination thereof in the subject,
wherein the at least one hormone and/or analyte is LH, progesterone, estrogen, or follicle stimulating hormone (FSH), or any metabolite thereof, or any combination of the foregoing.

29. The method of claim 28, further comprising:
using at least two urine-based LH test strips to indicate a time for use of the system, and wherein the system continuously measures at least LH for at least 6 hours.

30. The method of claim 29, wherein the system continuously measures at least LH for at least 24 hours.

31. The method of claim 28, further comprising:
taking at least one measurement of progesterone, or a metabolite thereof.

32. The method of claim 28, wherein the system continuously measures LH and progesterone for at least 6 hours.

33. The method of claim 28, further comprising:
providing continuous concentration data of at least one hormone and/or analyte measured, a predicted time of ovulation, a predicted time of peak fertility, or a confirmation that ovulation has occurred, or any combination thereof

34. The method of claim 28, further comprising:
providing continuous concentration data of at least one hormone and/or analyte measured, a predicted time of ovulation, a predicted time of peak fertility, and a confirmation that ovulation has occurred, or any combination thereof.

35. A method for predicting ovulation in a subject using a system of claim 1, the method comprising:
determining a baseline luteinizing hormone level in the subject prior to ovulation by continuous monitoring average luteinizing hormone levels over an initial time period of at least 10 minutes, wherein the initial time period is cycle start date until 18 days before the next expected cycle start date; and
predicting ovulation when the luteinizing hormone levels: a) increase at least 2-fold over the baseline luteinizing hormone level; and/or b) reach an absolute luteinizing hormone level of greater than 20 mUI/ml.

36. The method of claim 35, wherein ovulation is predicted when the luteinizing hormone levels increase 2-fold or 3-fold over the baseline luteinizing hormone level.

37. A method of confirming ovulation in a subject using a system of claim 1, the method comprising:
determining a baseline of progesterone level, or a level of metabolites thereof, in the subject prior to ovulation by continuous monitoring average progesterone levels, or average levels of metabolites thereof, over an initial time period of at least 10 minutes, wherein the initial time period is cycle start date until 18 days before the next expected cycle start date; and
confirming ovulation when progesterone levels, or levels of metabolites thereof: a) increase at least 2-fold over the baseline of progesterone level, or a level of metabolites thereof, and/or b) reach an absolute progesterone level, or level of metabolites thereof, of greater than 5 ng/ml progesterone or a metabolite thereof.

38. The method of claim 37, wherein ovulation is predicted when the progesterone levels, or levels of metabolites thereof, increase 2-fold or 3-fold over the baseline progesterone level, or level of metabolites thereof.

39. The method of claim 28, wherein the metabolite of progesterone is pregnanediol.

40. The method of claim 35, wherein the initial time period is at least 2 hours, or about 2 hours.

41. A method of confirming pregnancy and monitoring the health of a pregnancy in a subject using a system of claim 1, the method comprising:
measuring HCG, progesterone or a metabolite thereof, and/or luteinizing hormone (LH); and
a) calculating a conception date based on 1) the date in which LH was above 20 mUI/ml, 2) LH levels reach 2- or 3-fold above baseline values, 3) the date progesterone or a metabolite thereof levels reached 2-fold over baseline values, and/or 4) the range of dates between LH rise and rise in progesterone or a metabolite thereof as a possible conception window; and/or
b) assessing health of pregnancy by calculating 1) the rate at which HCG increases, 2) measuring progesterone or a metabolite thereof levels continuously to determine the rate at which progesterone or a metabolite thereof levels increase, to alert the subject when levels of progesterone or a metabolite thereof fall below 5 ng/ml, 10 ng/ml, or 15 ng/ml, and/or 3) drop in progesterone signals need to support pregnancy with additional progesterone supplementation.
